(19) United States
(12) Patent Application Publication
(10) Pub. No.: US 2024/0312639 A1
(43) Pub. Date: Sep. 19, 2024

(54) DEEP LEARNING FOR MODELING DISEASE PROGRESSION

(71) Applicant: Genentech, Inc., South San Francisco, CA (US)

(72) Inventors: Seyed Mohammadmohsen HEJRATI, South San Francisco, CA (US); Somaye Sadat HASHEMIFAR, South San Francisco, CA (US); Claudia IRIONDO, South San Francisco, CA (US)

(21) Appl. No.: 18/664,892

(22) Filed: May 15, 2024

Related U.S. Application Data
(63) Continuation of application No. PCT/US2022/079839, filed on Nov. 14, 2022.
(60) Provisional application No. 63/279,606, filed on Nov. 15, 2021.

(51) Int. Cl. G16H 50/50; G16H 50/20

(52) U.S. Cl. CPC G16H 50/50; G16H 50/20

(57) ABSTRACT

A method is provided for deep learning for modeling disease progression. The method may include generating, by a machine learning model, a first feature representation based on clinical data associated with a baseline cognitive state of a patient. The method may also include generating, by the machine learning model, a second feature representation based on an image of a brain of the patient. The method may also include generating, by the machine learning model, a set representation by at least fusing the first feature representation and the second feature representation. The method may also include predicting, by the machine learning model, a change in the baseline cognitive state over a time period based at least on the set representation. Related systems and articles of manufacture are also disclosed.

### CROSS-REFERENCE TO RELATED APPLICATIONS

The present application claims priority to U.S. Provisional Application No. 63/279,606, filed Nov. 15, 2021, and entitled, “MODELING THE PROGRESSION OF A DISEASE WITH A MULTI-MODAL NEURAL NETWORK,” the entirety of which is incorporated herein by reference.

### FIELD

The present disclosure generally relates to machine learning and more specifically to deep learning for modeling disease progression.

### BACKGROUND

Alzheimer's disease is the most common cause of dementia in people over 65, with 26.6 million people suffering worldwide. Alzheimer's disease, like some other neurological disorders, is a slowly progressing disease caused by the degeneration of brain cells, with patients showing clinical symptoms years after the onset of the disease. Therefore, accurate diagnosis and treatment of Alzheimer's disease, among other neurological disorders, in its early stage (e.g., mild cognitive impairment (MCI)), can help to prevent non-reversible and fatal brain damage. Despite demand, little progress has been made in predicting progression of neurological disorders, such as Alzheimer's disease, because of the complexity in modeling the progression, lack of large-scale, homogeneous datasets that contain early stage Alzheimer's disease patients, and noisy endpoints that can be generally difficult to predict.

### SUMMARY

Methods, systems, and articles of manufacture, including computer program products, are provided for deep learning for modeling disease progression. In one aspect, there is provided a system. The system may include at least one data processor and at least one memory. The at least one memory may store instructions that result in operations when executed by the at least one data processor. The operations may include: generating, by a machine learning model, a first feature representation based on clinical data associated with a baseline cognitive state of a patient. The operations may also include generating, by the machine learning model, a second feature representation based on an image of a brain of the patient. The operations may also include generating, by the machine learning model, a set representation by at least fusing the first feature representation and the second feature representation. The operations may also include predicting, by the machine learning model, a change in the baseline cognitive state over a time period based at least on the set representation.

In another aspect, there is provided a method for deep learning for modeling disease progression. The method may include: generating, by a machine learning model, a first feature representation based on clinical data associated with a baseline cognitive state of a patient. The method may also include generating, by the machine learning model, a second feature representation based on an image of a brain of the patient. The method may also include generating, by the machine learning model, a set representation by at least fusing the first feature representation and the second feature representation. The method may also include predicting, by the machine learning model, a change in the baseline cognitive state over a time period based at least on the set representation.

In another aspect, there is provided a computer program product that includes a non-transitory computer readable storage medium. The non-transitory computer-readable storage medium may include program code that causes operations when executed by at least one data processor. The operations may include: generating, by a machine learning model, a first feature representation based on clinical data associated with a baseline cognitive state of a patient. The operations may also include generating, by the machine learning model, a second feature representation based on an image of a brain of the patient. The operations may also include generating, by the machine learning model, a set representation by at least fusing the first feature representation and the second feature representation. The operations may also include predicting, by the machine learning model, a change in the baseline cognitive state over a time period based at least on the set representation.

In some variations of the methods, systems, and non-transitory computer readable media, one or more of the following features can optionally be included in any feasible combination.

In some variations, the fusing is performed using one or more fusion techniques including at least one of: concatenation, summation, simple attention, scaled dot product attention, applying a tensor fusion network, low rank fusion, and unidirectional contextual attention.

In some variations, the first feature representation is an encoded vector including a concatenation of at least one of a current cognitive score representing the baseline cognitive state of the patient, demographic information associated with the patient, and genomic information associated with the patient.

In some variations, the second feature representation includes at least one domain invariant embedded feature.

In some variations, the machine learning model includes a first machine learning model trained to generate the first feature representation, a second machine learning model trained to generate the second feature representation, a third machine learning model trained to generate the set representation, and a fourth machine learning model trained to predict the change in the baseline cognitive state over the time period.

In some variations, the machine learning model is trained, based at least on a plurality of modalities including the clinical data associated with the baseline cognitive state of the patient and the image of the brain of the patient.

In some variations, the machine learning model is pre-trained to predict the baseline cognitive state of the patient based at least on a plurality of brain images acquired at a plurality of time points and across a plurality of domains.

In some variations, the machine learning model is trained by at least adversarially training a domain detector of the machine learning model to reduce an inter-study domain shift associated with the image of the brain of the patient.

In some variations, the adversarially training includes adversarially training a feature extraction network of the machine learning model to learn domain invariant features for generating the second feature representation based at least on the image of the brain of the patient.

In some variations, the adversarially training includes applying a reverse gradient to the second feature representation to generate a domain detector input. The adversarially training the domain detector is based at least on the domain detector input.

In some variations, the domain detector indicates a drift in the inter-study domain shift at inference.

In some variations, the change in the baseline cognitive state over time indicates a progression of Alzheimer's disease in the patient.

In some variations, the time period is 12 months.

In some variations, the image is a three-dimensional magnetic resonance imaging image including an inferred mask.

In some variations, the baseline cognitive state is represented by at least one cognitive score including at least one of a Clinical Dementia Rating Scale Sum of Boxes (CDRSB) score, an Alzheimer's Disease Assessment Scale-Cognitive Subscale (ADAS-COG12) score, and a Mini-Mental State Examination (MMSE) score.

In some variations, a system is provided for generating an output indicating a prediction of the progression of the disease in the subject. The system performs operations including receiving longitudinal physiological data representative of a subject, the longitudinal physiological data including a plurality of modalities indicative of a progression of a disease in the subject. The system also performs operations including applying a trained multi-modal neural network to the longitudinal physiological data. The trained multi-modal neural network includes a plurality of feedforward neural networks configured to learn modality-specific deep features related to the plurality of modalities. The system also performs operations including generating an output indicating a prediction of the progression of the disease in the subject based at least on the modality-specific deep features related to the plurality of modalities of the longitudinal physiological data.

In some variations, the operations further include performing a plurality of domain shifts on the modality-specific deep features, the plurality of domain shifts configured to normalize the modality-specific deep features to a single domain, combining the normalized modality-specific deep features to form a feature space in the single domain, the feature space indicative of the progression of the disease, and generating, based at least on the normalized modality-specific deep features in the feature space, the output indicating the prediction of the progression of the disease in the subject. Additionally, the operations further include debiasing each normalized modality-specific deep features of the normalized modality-specific deep features using an adversarial loss to alleviate a study-specific bias corresponding to a modality of the plurality of modalities.

In some variations, the operations further include applying a sharpness-aware minimization optimization technique to improve the plurality of domain shifts on the modality-specific deep features to the single domain. Further, each modality of the plurality of modalities are fed to a separate feedforward neural network of the plurality of feedforward neural networks. Additionally, the longitudinal physiological data includes baseline physiological data representative of the subject, the baseline physiological data including a first set of modalities indicative of the progression of the disease, the physiological data further including a baseline image related to a subject brain. Further, the longitudinal physiological data includes delta physiological data representative of the subject, the delta physiological data including a second set of modalities indicative of the progression of the disease, the delta physiological data further including a delta image related to the subject brain. In some variations, the trained multi-modal neural network includes a feature extraction network, an endpoint prediction network, and a domain detector. Additionally, the disease is Alzheimer's disease and wherein the output is a normalized representation.

In another aspect, computer-readable storage mediums are provided for generating an output indicating a prediction of the progression of the disease in the subject. The computer-readable storage mediums include instructions including receiving longitudinal physiological data representative of a subject, the longitudinal physiological data including a plurality of modalities indicative of a progression of a disease in the subject. The computer-readable storage mediums also include instructions including applying a trained multi-modal neural network to the longitudinal physiological data. The trained multi-modal neural network includes a plurality of feedforward neural networks configured to learn modality-specific deep features related to the plurality of modalities. The computer-readable storage mediums also include instructions including generating an output indicating a prediction of the progression of the disease in the subject based at least on the modality-specific deep features related to the plurality of modalities of the longitudinal physiological data.

In some variations, the instructions further include performing a plurality of domain shifts on the modality-specific deep features, the plurality of domain shifts configured to normalize the modality-specific deep features to a single domain, combining the normalized modality-specific deep features to form a feature space in the single domain, the feature space indicative of the progression of the disease, and generating, based at least on the normalized modality-specific deep features in the feature space, the output indicating the prediction of the progression of the disease in the subject. Additionally, the instructions further include debiasing each normalized modality-specific deep features of the normalized modality-specific deep features using an adversarial loss to alleviate a study-specific bias corresponding to a modality of the plurality of modalities.

In some variations, the instructions further include applying a sharpness-aware minimization optimization technique to improve the plurality of domain shifts on the modality-specific deep features to the single domain. Further, each modality of the plurality of modalities are fed to a separate feedforward neural network of the plurality of feedforward neural networks. Additionally, the longitudinal physiological data includes baseline physiological data representative of the subject, the baseline physiological data including a first set of modalities indicative of the progression of the disease, the physiological data further including a baseline image related to a subject brain. Further, the longitudinal physiological data includes delta physiological data representative of the subject, the delta physiological data including a second set of modalities indicative of the progression of the disease, the delta physiological data further including a delta image related to the subject brain. In some variations, the trained multi-modal neural network includes a feature extraction network, an endpoint prediction network, and a domain detector. Additionally, the disease is Alzheimer's disease and wherein the output is a normalized representation.

In yet another aspect, methods are provided for generating an output indicating a prediction of the progression of the disease in the subject. The methods include receiving longitudinal physiological data representative of a subject, the longitudinal physiological data including a plurality of modalities indicative of a progression of a disease in the subject. The methods also include applying a trained multi-modal neural network to the longitudinal physiological data. The trained multi-modal neural network includes a plurality of feedforward neural networks configured to learn modality-specific deep features related to the plurality of modalities. The methods also include generating an output indicating a prediction of the progression of the disease in the subject based at least on the modality-specific deep features related to the plurality of modalities of the longitudinal physiological data.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to deep learning for modeling disease progression, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts an example disease progression prediction system, consistent with implementations of the current subject matter;

FIG. 2A depicts an example progression of Alzheimer's disease, consistent with implementations of the current subject matter;

FIG. 2B depicts an example progression plot illustrating the progression of Alzheimer's disease in various patients, consistent with implementations of the current subject matter;

FIG. 3 depicts an example graph illustrating various measurements of the severity of Alzheimer's disease over time, consistent with implementations of the current subject matter;

FIG. 4A depicts a network diagram illustrating a disease progression prediction system, consistent with implementations of the current subject matter;

FIG. 4B depicts an example dense layer of the network diagram, consistent with implementations of the current subject matter;

FIG. 4C depicts an example transition block of the network diagram, consistent with implementations of the current subject matter;

FIG. 5 depicts a diagram of an example fusion technique, consistent with implementations of the current subject matter;

FIG. 6 depicts a flowchart illustrating an example of a process for deep learning for modeling disease progression, consistent with implementations of the current subject matter;

FIG. 7 depicts a table summarizing an example dataset for a disease progression prediction system, consistent with implementations of the current subject matter;

FIG. 8 depicts a table summarizing a performance comparison of machine learning models and approaches, consistent with implementations of the current subject matter;

FIG. 9 depicts a table summarizing a performance comparison of machine learning models and approaches, consistent with implementations of the current subject matter;

FIG. 10 depicts a graph illustrating a performance of a disease progression prediction system, consistent with implementations of the current subject matter;

FIG. 11 depicts a table summarizing a performance comparison of machine learning models and approaches, consistent with implementations of the current subject matter;

FIG. 12 depicts a graph illustrating a performance comparison of optimization approaches, consistent with implementations of the current subject matter;

FIG. 13 depicts a table summarizing a performance comparison of machine learning models and approaches, consistent with implementations of the current subject matter; and

FIG. 14 depicts a block diagram illustrating an example of a computing system, consistent with implementations of the current subject matter.

When practical, like labels are used to refer to same or similar items in the drawings.

### DETAILED DESCRIPTION

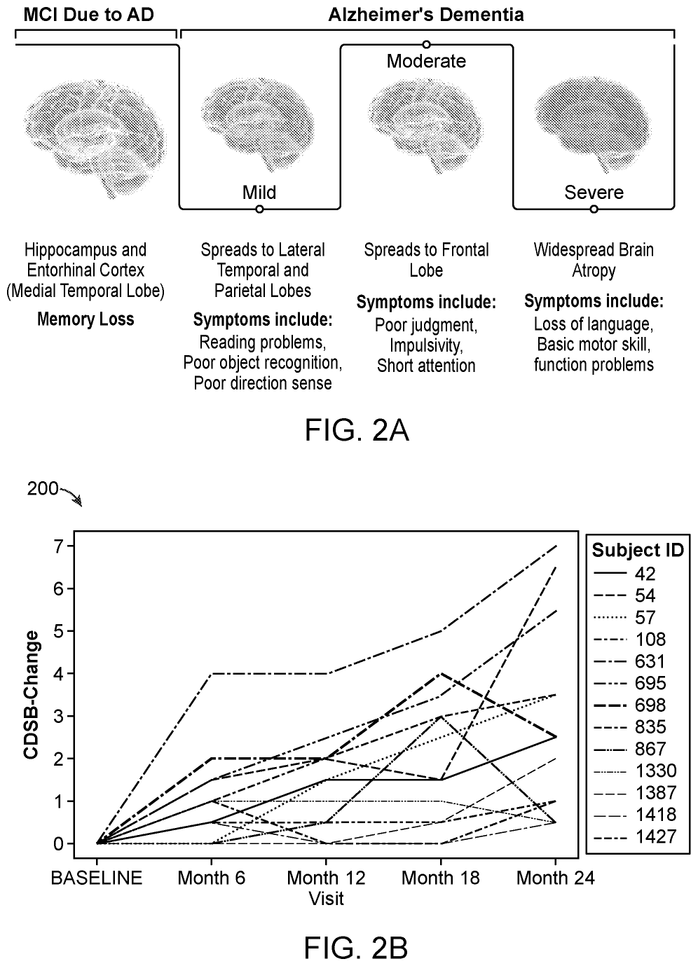

Diagnostic methods for diagnosing neurological disorders, such as Alzheimer's disease, have focused on the task of classifying patients into coarse categories, including Cognitive Normal (CN), Mild Cognitive Impairment (MCI), or Alzheimer's disease (AD), or to predict conversion from one class to another (e.g. MCI to AD). Diagnostic methods have further been used to classify patients into finer categories, such as MCI, mild Alzheimer's disease, Moderate Alzheimer's disease, and Severe Alzheimer's disease. As an example, as shown in FIG. 2A, MCI due to Alzheimer's disease may impact the hippocampus and entorhinal cortex of a brain of a patient, mild Alzheimer's disease in the patient may spread to the lateral temporal and parietal lobes of the brain of the patient, moderate Alzheimer's disease may spread to the frontal lobe of the brain of the patient, and severe Alzheimer's disease may exhibit widespread brain atrophy.

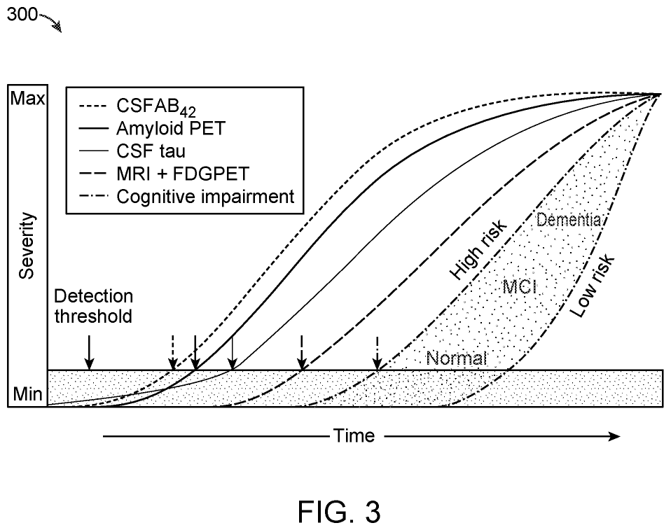

However, applications in clinical trials use a more fine-grained measurement scale, because clinical trial populations may generally be narrowly defined (e.g., only MCI, or the like). One approach is to instead predict the outcome of cognitive and functional tests, such as Clinical Dementia Rating Scale Sum of Boxes (CDRSB), Alzheimer's Disease Assessment Scale-Cognitive Subscale (ADAS-COG12), Mini-Mental State Examination (MMSE), and others, which are measured by continuous numerical values. For example, FIG. 3 shows a graph 300 illustrating various measurements of the severity of Alzheimer's disease over time. This approach helps to provide more granular estimations of disease progression, but can be very noisy and subjective across studies, making it challenging to establish treatment efficacy in small patient populations. Further, FIG. 2B depicts a graph 200 illustrating that even patients having the same diagnosis based on the predicted outcome of the cognitive and functional testing can have varying progression patterns. Thus, as patients with Alzheimer's disease may present different phenotypes and progression patterns, it can be beneficial to provide patients with personalized therapies and treatment plans. Also, predicting only the diagnosis does not generally provide fine-grained scale measurement about the progression of the disease.

In some instances, machine learning and deep learning based approaches have been used for diagnosing, monitoring, and treating patients having Alzheimer's disease. However, such approaches have focused on developing single-task and/or single modality models, which are not applicable to personalized medicine for Alzheimer's disease. Moreover, single-task and single-modality-based models exploit neither the complementary information among modalities nor the correlation between tasks, leading to less accurate predictions, diagnoses, and treatment plans.

Accordingly, accurate predictions of clinical trajectories for patients with Alzheimer's disease (AD) have the potential to increase the efficiency of evaluating novel therapeutics for this complex and heterogeneous disease. Predicted progression rates from prognostic models can be used for covariate adjustment in the primary analysis of a clinical trial to increase the precision of the treatment effect estimate and hence increase power. However, as noted, current machine learning approaches incorporate either single-task or single-modality models, which cannot make use of diverse and rich data such as high-dimensional neuroimaging and features of clinical data. Moreover, those approaches are generally trained on a single dataset (e.g., cohort), which cannot be easily generalized to other cohorts.

The machine learning model, consistent with implementations of the current subject matter, provides a multimodal approach to predicting progression in neurological diseases, such as Alzheimer's disease. For example, the machine learning model described herein accounts for multiple modalities, such as clinical data including environmental factors, genomics, demographics, and/or the like, and medical (e.g., brain) imaging, and/or the like, while modeling the complex interactions between each modality. Thus, as medical datasets grow in size and complexity, the machine learning model described herein is able to integrate various modalities to characterize and contextualize a patient's condition, and accurately and efficiently predict the progression of the patient's condition, such as the patient's cognitive state. With an accurate multimodal forward model of disease progression, the machine learning model consistent with implementations of the current subject matter accounts for variable rates of disease progression when assessing the effects of novel treatments.

As described herein, the deep learning approach to modeling disease progression includes a multimodal multi-task deep learning model that predicts disease (e.g., Alzheimer's disease) progression by, for example, analyzing longitudinal clinical and neuroimaging data from multiple cohorts. The described machine learning model integrates high dimensional magnetic resonance imaging features generated by, for example, a three-dimensional convolutional neural network, with other data modalities, including clinical data, to predict the progression (e.g., a change) in the disease in patients. As described herein, the machine learning model may employ an adversarial loss to alleviate the study-specific imaging bias, such as the inter-study domain shifts, between the sources of the clinical data and/or images. Additionally and/or alternatively, the machine learning model may employ a Sharpness-Aware Minimization (SAM) optimization technique to further improve generalization and reduce inter-study domain shifts. As described in more detail below, the machine learning model yields a significant improvement over and outperforms other models.

As an example, the machine learning model consistent with implementations of the current subject matter may generate a first feature representation based on a first modality, such as clinical data, associated with a baseline cognitive state of a patient. The machine learning model may generate a second feature representation based on a second modality, such as an image of a brain of the patient. The machine learning model may also generate a set representation by at least fusing the first feature representation and the second feature representation. The machine learning model may predict a change in the baseline cognitive state over a time period based at least on the set representation. Accordingly, the machine learning model accurately and efficiently predicts a change in the cognitive state of the patient. This allows for early and accurate diagnosing of diseases, such as neurological diseases including Alzheimer's disease in patients. Additionally and/or alternatively, the machine learning model described herein makes accurate predictions that can be used to provide an appropriate treatment plan for treating the diagnosed disease and/or based on the predicted progression of the disease in the patient.

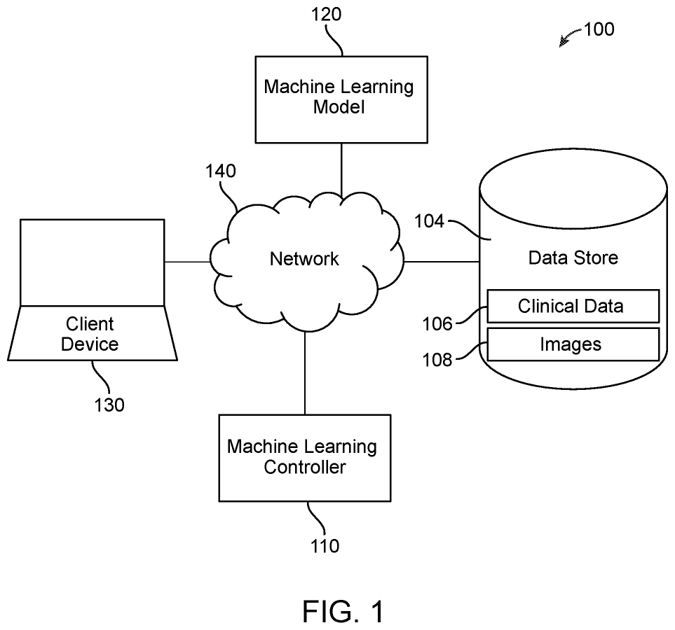

FIG. 1 depicts a system diagram illustrating an example of a disease progression prediction system 100, consistent with implementations of the current subject matter. Referring to FIG. 1, the disease progression prediction system 100 may include a machine learning controller 110, a machine learning model 120, a client device 130, and a data store 104. The machine learning controller 110, the machine learning model 120, the client device 130, and the data store 104 may be communicatively coupled via a network 140. The network 140 may be a wired network and/or a wireless network including, for example, a wide area network (WAN), a local area network (LAN), a virtual local area network (VLAN), a public land mobile network (PLMN), the Internet, and/or the like. In some implementations, the machine learning controller 110, the machine learning model 120, the data store 104, and/or the client device 130 may be contained within and/or operate on a same device.

It should be appreciated that the client device 130 may be a processor-based device including, for example, a smartphone, a tablet computer, a wearable apparatus, a virtual assistant, an Internet-of-Things (IoT) appliance, and/or the like. The client device 130 may form a part of, include, and/or be coupled to a magnetic resonance imaging machine.

Referring to FIG. 1, the data store 104 may store clinical data 106 and/or one or more images 108. The clinical data 106 and/or the one or more images 108 may define a multimodal input, from a plurality of modalities, for the machine learning model 120. The clinical data 106 may be associated with a baseline cognitive state of a patient. The baseline cognitive state of the patient may be represented by at least one cognitive score. Thus, the clinical data 106 may include the at least one cognitive score including at least one of a Clinical Dementia Rating Scale Sum of Boxes (CDRSB) score, an Alzheimer's Disease Assessment Scale-Cognitive Subscale (ADAS-COG12) score, a Mini-Mental State Examination (MMSE) score, a Functional Activities Questionnaire (FAQ) score, and/or the like.

Additionally and/or alternatively, the clinical data 106 includes demographic information associated with the patient, and/or genomic information associated with the patient. In some implementations, the demographic information includes at least one of an age, a sex, a diagnosis, an education level, a body mass index, and/or the like of the patient. The genomic information may include a presence or absence of Apolipoprotein E4 (APOE4), which is a risk factor gene for predicting Alzheimer's disease in the patient. The clinical data 106 may be collected at a baseline visit.

The clinical data 106 may be specific to a particular patient and/or may include data corresponding to a plurality of patients. For example, the clinical data 106 corresponding to a particular patient may be collected at a baseline visit. The clinical data 106 collected at the baseline visit may be used as an input modality to the machine learning model 120 to accurately predict the progression in the cognitive state of the patient. Additionally and/or alternatively, the clinical data 106 may correspond to one or more patients. The clinical data 106 may additionally and/or alternatively be collected at one or more time points. Thus, the clinical data 106 corresponding to the one or more patients and/or the one or more time points may be used by the machine learning controller 110 to train the machine learning model 120.

The clinical data 106 may be represented as a D length vector including a concatenation of one or more cognitive scores, genomic information and/or demographic information, such as at the baseline visit. The vector may be used by the machine learning model 120 to predict the smoothed change in the one or more cognitive scores at a time period after the baseline visit. The time period may be one year (i.e., 12 months), or another time period, such as 6 months, 18 months, 24 months, and/or the like.

The images 108 may include one or more images of a brain of the patient. The one or more images of the brain may be acquired by the magnetic resonance imaging machine. The one or more images 108 of the brain of the patient may include three-dimensional magnetic resonance images of the brain of the patient. For example, the three-dimensional magnetic resonance images may include a slice (a sagittal and/or a coronal slice) having one or more dimensions, such as a height (H), a width (W), a thickness (T), and channel (C). The one or more images 108 may be raw images and/or be pre-processed images. For example, the one or more images 108 may include a brain mask (e.g., an inferred mask) inferred for each magnetic resonance image volume. The brain mask may be generated by at least applying one or more image segmentation techniques to the magnetic resonance image volume. For example, the brain mask may be predicted based at least on intensity normalization performed based on a distribution of intensities of the brain in the image. The brain mask may be applied to the raw image of the brain of the patient. The brain mask may highlight the brain within the one or more images 108 and/or a particular portion of the brain within the one or more images 108. This allows for the machine learning model 120 to ignore portions of the one or more images 108 that do not correspond to the brain.

Consistent with implementations of the current subject matter, the machine learning controller 110 may include a processor and a memory storing instructions, which when executed by the processor perform one or more operations described herein. For example, as described herein, the machine learning controller 110 may train and/or implement the machine learning model 120, such as based on one or more modalities including the clinical data 106 and/or the images 108.

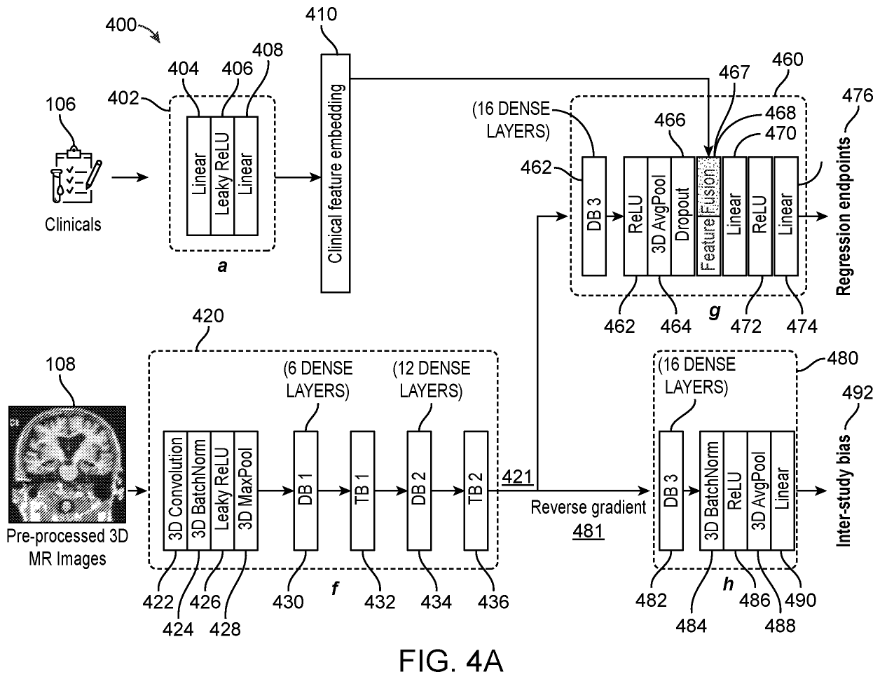

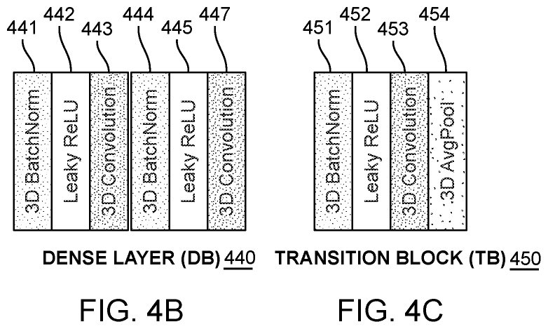

FIG. 4A schematically illustrates an example architecture 400 of the machine learning model 120. The machine learning controller 110 may train the machine learning model 120 using the architecture 400. As noted, the machine learning model 120 may include a multimodal multi-task deep neural network that combines three-dimensional magnetic resonance images (e.g., the images 108) and clinical features (e.g., the clinical data 106), including one or more cognitive scores, genomic information, and demographic information, into a single end-to-end model to predict the future status (e.g., a progression of disease and/or a change in cognitive state of a patient) of a patient from their baseline visit. Each modality (e.g., the clinical data 106 and/or the image 108) is fed into the machine learning model 120 through separate branches to learn modality-specific latent spaces, while leveraging complementary knowledge between the different modalities.

Referring to the architecture 400, the machine learning model 120 may include one or more machine learning models, such as one, two, three, four, or more machine learning models. The one, two, three, four, or more machine learning models may include separate networks and/or may form a part of a single machine learning model (e.g., the machine learning model 120). As shown in FIG. 4, the machine learning model 120 may include a first machine learning model or network, such as an encoder 402, a second machine learning model or network, such as feature extraction network 420, a third machine learning model or network, such as an endpoint prediction network 460, and/or a fourth machine learning model or network, such as a domain detector 480. In some implementations, the encoder 402, the feature extraction network 420, the endpoint prediction network 460, and the domain detector 480 may form a single machine learning model (e.g., the machine learning model 120). In other implementations, the encoder 402, the feature extraction network 420, the endpoint prediction network 460, and the domain detector 480 form separate, but connected, machine learning models. In yet other implementations, the encoder 402 defines a first machine learning model and the feature extraction network 420, the endpoint prediction network 460, and the domain detector 480 form subnetworks of a second machine learning model connected to the first machine learning model.

Consistent with implementations of the current subject matter, the machine learning model 120 includes a multimodal multi-task deep neural network for predicting disease progression and diagnosis. The machine learning model 120 leverages imaging and clinical data to generate a patient-specific progression prediction, and to generalize the predictions across several domains (e.g., different source datasets, such as inter-study datasets, and/or patient disease states). For example, the machine learning model 120 combines various inputs, including the clinical data 106 (e.g., the one or more cognitive scores, demographic information, and/or genomic information), and images 108 (e.g., the three-dimensional magnetic resonance brain images), which enables learning of complementary information across modalities for a large patient population. Tabular data and imaging data are fed (e.g., by the machine learning controller 110) as separate inputs to the machine learning model 120 for generating feature representations that are fused down-stream. As described in more detail below, the machine learning controller 110 may train the machine learning model 120 end to end to construct a feature space for predicting the progression in the cognitive state, as represented by a change in one or more cognitive scores of the patient.

Referring to FIG. 4A, the machine learning model 120 includes the encoder 402. The encoder 402 may receive the clinical data 106 associated with the baseline cognitive state of the patient. As described herein, the clinical data 106 may include one or more cognitive scores representing a current or baseline cognitive state of the patient, demographic information associated with the patient at a baseline visit, and/or genomic information associated with the patient at the baseline visit.

The baseline cognitive state of the patient may be predicted by the machine learning model 120. For example, the machine learning controller 110 may pre-train the machine learning model 120 to predict the baseline cognitive state of the patient, based at least on a plurality of brain images (e.g., the images 108) acquired at a plurality of time points and across a plurality of domains (e.g., clinical studies). This leverages longitudinal features of the images 108 acquired at all available time points for pre-training. Integrating the longitudinal features of the images 108 allows the machine learning model 120 to learn the underlying temporal characteristics of the target disease, such as Alzheimer's diseases. In this way, the machine learning controller 110 may pre-train the machine learning model 120 to predict the current cognitive scores (e.g., the baseline cognitive state) for each visit of the patient, such as at various time points. The machine learning controller 110 may use the pre-trained weights as a starting point for training the machine learning model 120 to predict the change in the baseline cognitive state (e.g., the baseline cognitive score) for the patient.

In some instances, the plurality of domains and/or the clinical data (e.g., the current cognitive scores, etc.) may introduce domain shift biases into the input (e.g., the images 108 and/or the clinical data 106) of the machine learning model 120. For example, with respect to the images 108, an image of a slice of a brain from one domain may vary significantly in appearance from an image of the same slice of the brain from another domain. As described in more detail below, the machine learning model 120 may be trained by, for example, the machine learning controller 110, to reduce or eliminate such domain shifts, and to improve the accuracy of predictions made by the machine learning model 120.

As shown in FIG. 4A, the encoder 402 may generate a first feature representation 410 based on the clinical data 106. The first feature representation 410 may include one or more embedded clinical features extracted from the clinical data 106, such as the baseline clinical data. The one or more embedded clinical features may be represented as an encoded vector including a concatenation of at least one of a current cognitive score representing the baseline cognitive state of the patient, demographic information associated with the patient, and genomic information associated with the patient.

Referring to FIG. 4A, the encoder 402 may include a plurality of layers. The plurality of layers may include a linear layer 404, a leaky ReLU layer 406, and a linear layer 408. The linear layer 404, the leaky ReLU layer 406, and the linear layer 408 may be stacked to improve extraction of the embedded clinical features defining the first feature representation 410.

Referring again to FIG. 4A, the machine learning model 120 includes the feature extraction network 420. The feature extraction network 420 may receive the image 108 associated with the baseline cognitive state of the patient. As described herein, the image 108 may include one or more baseline (e.g., current) brain images of the patient. The image 108 may be acquired at the baseline visit. The image 108 may include one or more baseline magnetic resonance images received from one or more domains (e.g., directly from a magnetic resonance imaging machine, a scanner, or the like).

The feature extraction network 420 may generate, as an output of the feature extraction network 420, a second feature representation 421 based on the image 108 of the brain of the patient. The second feature representation 421 may include one or more embedded features, such as one or more domain invariant embedded features (described in more detail below), extracted from the image 108. The one or more embedded features of the second feature representation 421 may include one or more pixel values, spatial-temporal features, and/or the like, extracted from the image 108. The second feature representation 421 may be outputted by the feature extraction network 420 and may be used, by the machine learning controller 110, as an input to the endpoint prediction network 460 and/or the domain detector 480.

The feature extraction network 420 includes one or more stacked layers, including a convolutional layer 422 (e.g., a three-dimensional convolutional layer), a batch normalization layer 424 (e.g., a three-dimensional batch normalization layer), a hidden layer 426 (e.g. a leaky ReLU layer), and a max-pooling layer 428 (e.g., a three-dimensional max-pooling layer). The feature extraction network 420 may include one or more dense layers or blocks 440 (see FIG. 4B) and/or one or more transitional blocks 450 (see FIG. 4C) following the one or more stacked layers.

The one or more dense layers 440 may include a first dense layer 430, a second dense layer 434, a third dense layer 462, a fourth dense layer 482, and/or the like. Referring to FIG. 4B, each of the one or more dense layers 440 includes six dense layers (e.g., two sets of stacked three dense layer blocks) or twelve dense layers (e.g., four sets of stacked three dense layer blocks). For example, each set of stacked three dense layer blocks includes a batch normalization layer, a hidden layer, and a convolutional layer. As shown in FIG. 4B, the dense layer 440 includes six dense layers, such as a batch normalization layer 441 (e.g., a three-dimensional batch normalization layer), a hidden layer 442 (e.g., a leaky ReLU layer), a convolutional layer 443 (e.g., a three-dimensional convolutional layer), a batch normalization layer 444 (e.g., a three-dimensional batch normalization layer), a hidden layer 445 (e.g., a leaky ReLU layer), and a convolutional layer 447 (e.g., a three-dimensional convolutional layer). The first dense layer 430 includes six dense layers, such as two sets of stacked three dense layer blocks). The second dense layer 434 includes twelve dense layers, such as four sets of stacked three dense layer blocks. The third and fourth dense layers 462, 482 include sixteen stacked dense layer blocks. Each of the one or more dense layers generates a feature map by extracting one or more features from the image 108 after the image 108 has passed through a previous block.

Referring to FIG. 4A, the one or more transition blocks 450 may include a first transition block 432 and a second transition block 436. Referring to FIG. 4B, each of the first transition blocks 450 includes four dense layers, such as a batch normalization layer 451 (e.g., a three-dimensional batch normalization layer), a hidden layer 452 (e.g., a leaky ReLU layer), a convolutional layer 453 (e.g., a three-dimensional convolutional layer), and an average pooling layer 454 (e.g., a three-dimensional average pooling layer). Each of the one or more layers of the one or more transition blocks 450 generates a feature map by extracting one or more features from the image 108 after the image 108 has passed through a previous block.

Referring back to FIG. 4A, the feature extraction network 420 includes the one or more stacked layers, followed by the first dense layer 430, the first transition block 432, the second dense layer 434, and the second transition block 436. The feature map size changes between the first and second dense layers 430, 434 and through the first and second transition blocks 432, 436. As noted, the feature extraction network 420 generates a second feature representation including one or more extracted features based on the image 108.

The machine learning controller 110 provides the second feature representation 421 as an input to the endpoint prediction network 460 and the domain detector 480. The endpoint prediction network 460 includes the dense layer 462, a hidden layer 463 (e.g., an ReLU layer), an average pooling layer 464 (e.g., a three-dimensional average pooling layer), a dropout layer 466, a feature fusion layer 468, a linear layer 470, a hidden layer 472 (e.g., an ReLU layer), and/or a linear layer 474.

The endpoint prediction network 460 may generate one or more regression endpoints 476. For example, the endpoint prediction network 460 may predict a change in the baseline cognitive state over a time period (e.g., 12 months from baseline, 24 months from baseline, 48 months from baseline, etc.). For example, the change in the baseline cognitive state may be represented as a change in the one or more cognitive scores. The change in the baseline cognitive state over time may indicate the progression of the corresponding neurological disorder, such as Alzheimer's disease, in the patient.

As noted, the endpoint prediction network 460 includes the feature fusion layer 468. While the feature fusion layer 468 is shown as being included in the endpoint prediction network 460, the feature fusion layer 468 may form at least a part of one or more of the encoder 402, the feature extraction network 420, the domain detector 480, the endpoint prediction network 460, or another location as part of the machine learning model 120.

The feature fusion layer 468 may generate a set representation 467 by at least fusing the first feature representation 410 from the encoder 402 and/or the second feature representation 421 form the feature extraction network 420. The feature fusion layer 468 may fuse the first feature representation 410 and the second feature representation 421 using one or more fusion techniques, including concatenation, summation, simple attention, scaled dot product attention, applying a tensor fusion network, low rank fusion, unidirectional contextual attention, and/or the like.

As an example, the machine learning model 120, is trained to extract the first feature representation 410 and the second feature representation 421 from different modalities (e.g., the clinical data 106 and the image 108, respectively). Multimodal fusion may be achieved by the feature fusion layer 468 by, for example, one or more joint representation techniques including concatenating and/or summing the first feature representation 410 and the second feature representation 421 to generate the set representation 467. The set representation 467 is then fed to the fully connected layers of the endpoint prediction network 460 to predict the change in the cognitive state of the patient. In this example, gradients are backpropagated through the fully connected layers and upstream to each branch of the machine learning model 120, training the machine learning model 120 end-to-end. Thus, joint representation learning can leverage complementary information across modalities (e.g., the clinical data 106 and the image 108) and model inter-modality interactions. In some implementations, prior to generation of the set representation 467 at the feature fusion layer 468, the first feature representation 410 and/or the second feature representation 421 are normalized so that the first feature representation 410 and/or the second feature representation 421 have the same scale.

In some implementations, the set representation 467 may be generated using simple attention. In simple attention, the input first feature representation 410 and the second feature representation 421 are concatenated and fed through a single block, including two fully connected layers, an ReLU layer, and/or a sigmoid, using a nonlinear combination of features within and between the modalities (e.g., the clinical data 106 and/or the image 108) to reweight the concatenated feature vector of the set representation 467.

In some implementations, the set representation 467 may be generated using scaled dot product attention. In scaled dot product attention, the input first feature representation 410 and the second feature representation 421 are first concatenated into a single vector, then used as a query, key, and value in a scaled dot product computation. In some implementation, the machine learning controller 110 may instantiate one or more weight matrices of the input first feature representation 410 and the second feature representation 421 to linearly transform each query, key, and value to generate the set representation 467.

In some implementations, the set representation 467 may be generated using a tensor fusion network fusion technique. The feature fusion layer 468 may determine the tensor output between the first feature representation 410 and the second feature representation 421.

In some implementations, the set representation 467 may be generated using low-rank fusion: Low-rank fusion decomposes the input tensor including the first feature representation 410 and/or the second feature representation, and the learned weight tensor into low-rank factors. The feature fusion layer 468 then rearranges the decomposed input tensor to generate the set representation 467.

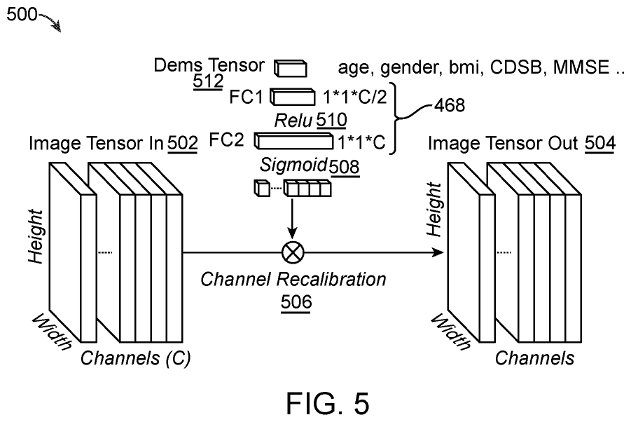

In some implementations, the set representation 467 may be generated using unidirectional contextual attention. FIG. 5 shows an example network diagram 500 showing the feature fusion layer 468. In this example, the feature fusion layer 468 assigns a primary modality from the plurality of modalities (e.g., the clinical data 106 and/or the image 108) and one or more auxiliary data modalities remaining from the plurality of modalities (e.g., the clinical data 106 and/or the image 108) to create a directed attention mechanism. The feature representation from the one or more auxiliary modalities may be input to the feature fusion layer 468 at 512 and concatenated and passed through the feature fusion layer 468, which may include two fully connected layers, such as a ReLU layer 510, and a sigmoid 508. The output from the feature fusion layer 468 (e.g., the sigmoid) is used to reweight the feature representation 502 at channel recalibration 506, generated based on the primary modality, to generate a reweighted feature representation 504.

As an example, the clinical data 106 may be an auxiliary modality and the image 108 may be a primary modality (or vice versa). In this example, the first feature representation 410 (e.g., input at 512) may pass through the fully connected layers 510, 508 of the feature fusion layer 468. The output may be used by the machine learning controller 110 (e.g., the machine learning model 120) at channel recalibration 506 to reweight the second feature representation 421 (e.g., feature representation 502 in this example) generated based on the image 108. The reweighted feature representation (e.g., the reweighted first feature representation 410 and/or the reweighted second feature representation 421 shown as feature representation 504 in FIG. 5) may define the set representation 467 generated and used as an input to the endpoint prediction network 460. In this way, the feature representation generated based on the auxiliary modalities (e.g., the clinical data 106 in this example) provide context to the feature representation generated based on primary modalities (e.g., the image 108 in this example) without dominating the optimization. Accordingly, the first feature representation 410 and the second feature representation 421 may be efficiently fused to generate the set representation 467 for predicting the one or more endpoints (e.g., the change in cognitive state, the regression endpoints 476, etc.) with improved accuracy.

Referring back to FIG. 4A, the domain detector 480 includes the dense layer 482, a batch normalization layer 484 (e.g., a three-dimensional batch normalization layer), a hidden layer 486 (e.g., a leaky ReLU layer), an average pooling layer 488 (e.g., a three-dimensional average pooling layer), and a linear layer 490.

The domain detector 480 allows the machine learning model 120 to learn domain invariant imaging feature representations, such as the domain invariant embedded features of the second feature representation 421. Generally, convolutional neural networks trained on multi-study images (e.g., magnetic resonance imaging data) often suffer from problems arising from domain shift and heterogeneity, as scanners and protocols may vary by study site and the patient population and their disease state may differ between studies. In particular, there are two common forms of domain shifts (e.g., biases) in medical image analysis among different studies: population shift and acquisition shift. The population shift occurs when cohorts of subjects exhibit varying demographic or clinical characteristics, while the acquisition shift is observed due to differences in imaging protocols, modalities or scanners. Thus, the image 108 may introduce domain shifting due to the population shift and/or acquisition shift. To alleviate possible inter-study biases, the machine learning controller 110 trains the machine learning model 120 to minimize the mutual information shared between the extracted feature and inter-study distribution shift, and to minimize bias 492, such as inter-study bias in the generated feature representations (e.g., the first feature representation 410 and/or the second feature representation 421) and/or regression endpoints 476.

In particular, the machine learning controller 110 adversarially trains the domain detector 480 against the feature extraction network 420 to predict the bias distribution (e.g., the bias 492). In other words, the machine learning controller 110 may train the machine learning model 120 using adversarial loss to extract neuroimaging features (e.g., the second feature representation 421) based on the images 108 and/or embedded clinical features (e.g., the first feature representation 410), while remaining invariant to study site domain. As a result of the adversarial training, the ability of the domain detector 480 to predict the domain associated with the image 108 is minimized.

For example, the feature extraction network 420 and/or the domain detector 480 are trained in an adversarial manner such that the feature extraction network 420 learns domain invariant features for generating the second feature representation 421. The feature extraction network 420 is trained adversarially to increase the ability of the feature extraction network 420 to generalize across multiple domains when generating the second feature representation 421. Accordingly, the feature extraction network 420 may be trained (e.g., by the machine learning controller 110) until the domain detector 480 is unable to correctly identify the domain of the second feature representation 421 generated by the feature extraction network 420.

As noted, the machine learning controller 110 adversarially trains the domain detector 480 to reduce an inter-study and/or inter modality domain shift associated with the image 108 of the brain of the patient. For example, the machine learning controller 110 trains the domain detector 480 to differentiate between the domains associated with the image 108 and/or the clinical data 106, based at least on the first feature representation 410 and/or the second feature representation 421. At the same time, the machine learning controller 110 trains the feature extraction network 420 to generalize the domain associated with the image 108 in generating the second feature representation 421, and/or the encoder 402 to generalize the domain associated with the clinical data 106 in generating the first feature representation 410. While the domain detector 480 is trained to minimize the domain classification loss, the feature extraction network 420 is trained to maximize the domain classification loss.

The machine learning controller 110 may adversarially train the domain detector 480 by, for example, applying a reverse gradient 481 to the second feature representation 421 (and/or the first feature representation 410) to generate the input to the domain detector 480. As noted, adversarially training the domain detector 480 results in the second feature representation 421 and/or the first feature representation 410 being domain invariant. In other words, the domain associated with the second feature representation 421 (or the first feature representation 410) is generalized such that there is a minimal or eliminated impact of the domain on the extracted features defining the second feature representation 421 (or the first feature representation 410). In some implementations, the domain detector 480 may additionally and/or alternatively be adversarially trained against the encoder 402 to minimize bias in the clinical data 106 and/or the first feature representation 410. Thus, the domain detector 480 may be adversarially trained to result in a domain invariant first feature representation 410 and/or a domain invariant second feature representation 421. At inference, the domain detector 480 may additionally and/or alternatively detect a drift in the bias 492.

As noted, the machine learning controller 110 may train the machine learning model 120, based at least on a plurality of modalities including the clinical data 106 associated with the baseline cognitive state of a patient p and the image 108 of the brain of the patient p, to predict a change in the baseline cognitive state over time. The input patient p with clinical features Clinₚ (e.g., the clinical data 106) and a 3D magnetic resonance imaging MRIₚ (e.g., the image 108) is fed as an input to the machine learning model 120, where Clinₚ enters the encoder 102 and MRIₚ enters the feature extraction network 420 to respectively learn clinical embeddings a(Clinₚ) (e.g., the first feature representation 410) and image embeddings f(MRIₚ) (e.g., the second feature representation 421) of the patient. Subsequently, f(MRIₚ) is fed into the domain detector 480 while both extracted features, a(Clinₚ) and f(MRIₚ), are fed forward through the endpoint prediction network 460. The parameters of each network are de-fined as θₐ (e.g., corresponding to the encoder 402), θ_{f} (e.g., corresponding to the feature extraction network 420), θ_{g} (e.g., corresponding to the endpoint prediction network 460), θₕ (e.g., corresponding to the domain detector 480), with the subscripts indicating their specific network as shown in FIG. 4A.

The machine learning model 120 performs robustly on test data from an unseen domain, even though the machine learning model 120 is trained based on a mixture of data sources. To this end, mutual information-based loss is added to the objective function for training the machine learning model 120. Therefore, the training procedure is to optimize the following equation:

$$Example 1 \\ min \text{?} {\sum }_{p} loss ( {y}_{endpoint} ( p ) , g ( f ( {MRI}_{p} ) ) + a ( {Clin}_{p} ) ) ) + λ I ( {y}_{bias} ( p ) ) ; \\ f ( {MRI}_{p} ) ) \qquad ( 1 )$$

$$\text{?} \text{indicates text missing or illegible when filed}$$

where loss(.,.) and I(.,.) respectively indicate the loss function and the mutual information (e.g., the bias 492), and λ is a hyper-parameter to balance the terms. Replacing the mutual information, the above equation becomes the following equation:

$$\begin{matrix}Equation 2 & \\ {min}_{{θ}_{a} , {θ}_{f} , {θ}_{g}} {max}_{{θ}_{h}} {\sum }_{p} [ \text{} {L}_{endpoint} ( p ) - μ {L}_{bias} loss ( {y}_{bias} ( p ) , h ∘ f ( {MRI}_{p} ) ) + λ H ( h ∘ f ( {MRI}_{p} ) ) ] & ( 2 )\end{matrix}$$

$${L}_{endpoint} ( p ) = loss ( {y}_{endpoint} ( p ) , g ( f ( {MRI}_{p} ) + a ( {Clin}_{p} ) ) )$$

$${L}_{bias} ( p ) = loss ( {y}_{bias} ( p ) , h ∘ f ( {MRI}_{p} ) )$$

$$H ( h ∘ f ( x ) ) = {\sum }_{x} h ∘ f ( x ) \times log ( h ∘ f ( x ) )$$

where L_{endpoint}(.) L_{bias}(.), and H(h∘f(.)) respectively represent the loss of the endpoint prediction, the loss of the bias prediction, and the entropy of the bias which acts as a regularizer. The set of networks, including the encoder 402, the feature extraction network 420, the endpoint prediction network 460, and the domain detector 480, are trained end to end, with the adversarial loss and gradient reversal technique (e.g., based on the reverse gradient 481), updating the weights θ_{f}θₕ. Early in learning, g∘f is rapidly trained to predict the endpoints by using the bias 492. Then, h (e.g., the domain detector 480) learns to predict the bias 492, and f (e.g., the feature extraction network 420) learns to extract feature embeddings (e.g., the second feature representation 421) invariant to the imaging domain.

Further, during training of the machine learning model 120, the machine learning controller 110 may apply sharpness-aware minimization (SAM) to avoid overfitting and improve generalization. SAM performs two forward-backward passes to estimate the smoothness of the loss landscape and improve final prediction accuracy. Smoothing is performed by at least fitting a per-patient linear regression on each cognitive score for all visits in the first 24 months (or another time period). The slope of the fitted linear regression is then used to determine the smoothed change in the cognitive scores (e.g., the cognitive state of the patient) at the end of a desired time period (e.g., 12 months). Training the machine learning model 120 using the smoothed change of cognitive scores helps to mitigate missing values and measurement noise.

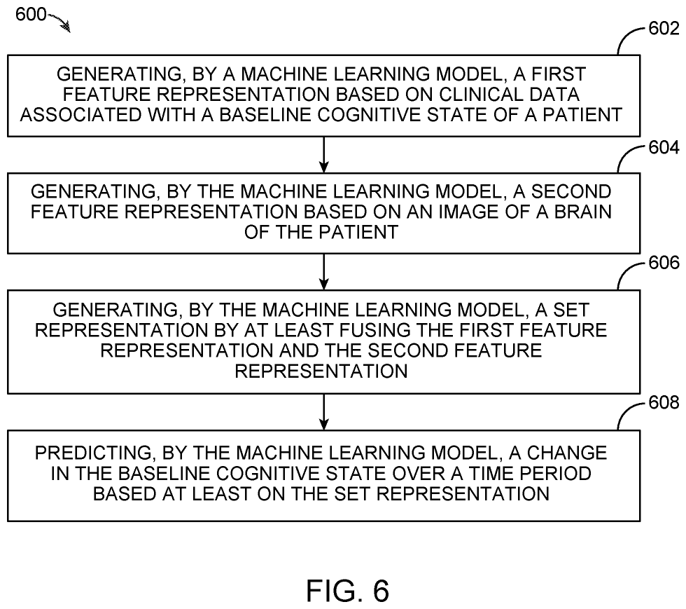

FIG. 6 depicts a flowchart illustrating an example of a process 600 for deep learning for modeling disease progression, such as the progression of one or more neurological disorders including Alzheimer's disease, consistent with implementations of the current subject matter. Referring to FIG. 6, the process 600 may be performed by the machine learning model 120 and/or the machine learning controller 110 to predict a change in a cognitive state of a patient over a time period, such as 12 months. This allows for efficient and accurate prediction of a progression of the neurological disorder, such as Alzheimer's disease, in the patient over time.

As described herein, the prediction may be domain invariant. In other words, the machine learning model 120 (e.g., the machine learning controller 110) may reduce an impact of domain shift biases stemming from an input (e.g., the image 108 and/or the clinical data 106) received from one or more modalities (e.g., one or more scanners, machines, studies, clinicians, etc.). Accordingly, the machine learning controller 110 may train the machine learning model 120 using multimodal modalities, including the clinical data 106 and/or the image 108, with improved accuracy, memory efficiency, and speed. Consistent with implementations of the current subject matter, the process 600 refers to the example architecture 400 shown in FIG. 4A.

The machine learning model 120 may receive one or more input modalities, such as the clinical data 106 associated with the baseline cognitive state of the patient and the image 108 of the brain of the patient. The image 108 may include a three-dimensional magnetic resonance image including an inferred mask. The baseline cognitive state may be represented by at least one cognitive score including at least one of a Clinical Dementia Rating Scale Sum of Boxes (CDRSB) score, an Alzheimer's Disease Assessment Scale-Cognitive Subscale (ADAS-COG12) score, and a Mini-Mental State Examination (MMSE) score.
At 602, the machine learning model 120 (e.g., via the machine learning controller 110) may generate a first feature representation, such as the first feature representation 410 based on clinical data, such as the clinical data 106 associated with a baseline cognitive state of a patient. The first feature representation 410 may be an encoded vector including a concatenation of at least one of a current cognitive score representing the baseline cognitive state of the patient, demographic information associated with the patient, and genomic information associated with the patient.

In some implementations, the machine learning model 120 may be pre-trained (e.g., by the machine learning controller 110) to predict the baseline cognitive state of the patient based at least on a plurality of brain images acquired at a plurality of time points and across a plurality of domains. The machine learning model 120 may be trained to reduce the inter-study domain shift associated with the plurality of domains. For example, the machine learning model 120 may be trained (e.g., by the machine learning controller 110) by at least adversarially training a domain detector (e.g., the domain detector 480) of the machine learning model 120 to reduce an inter-study domain shift (e.g., the bias 492) associated with at least the image 108 associated with the brain of the patient. The adversarially training includes applying a reverse gradient to the second feature representation 421 to generate a domain detector input to the domain detector 480. The adversarially training the domain detector 480 is based at least on the domain detector input. Further, as noted, the domain detector 480 may indicate a drift in the inter-study domain shift at inference. The adversarially training may include training the feature extraction network 420 in an adversarial manner such that the feature extraction network 420 learns domain invariant features for generating the second feature representation 421. In other words, the feature extraction network 420 may be trained adversarially to increase the ability of the feature extraction network 420 to generalize across multiple domains when generating the second feature representation 421.
At 604, the machine learning model 120 (e.g., via the machine learning controller 110) may generate a second feature representation, such as the second feature representation 421 based at least on an image, such as the image 108, of the brain of the patient. The second feature representation 421 may include at least one domain invariant embedded feature, as described herein. This helps to provide predictions with increased accuracy.
At 606, the machine learning model 120 (e.g., via the machine learning controller 110) may generate a set representation by at least fusing the first feature representation 410 and the second feature representation 421. The machine learning model 120 may perform the fusing using one or more fusion techniques. The one or more fusion techniques may include at least one of concatenation, summation, simple attention, scaled dot product attention, applying a tensor fusion network, low rank fusion, and unidirectional contextual attention.
At 608, the machine learning model 120 (e.g., via the machine learning controller 110) may predict a change in the baseline cognitive state over a time period based at least on the set representation. The change in the baseline cognitive state over time may indicate a progression of a disease, such as Alzheimer's disease in the patient. The time period may be 12 months, 24 months, 48 months, and/or the like. Accordingly, the machine learning model 120 may accurately and efficiently predict the progression of disease in the patient over time.

### Experiments

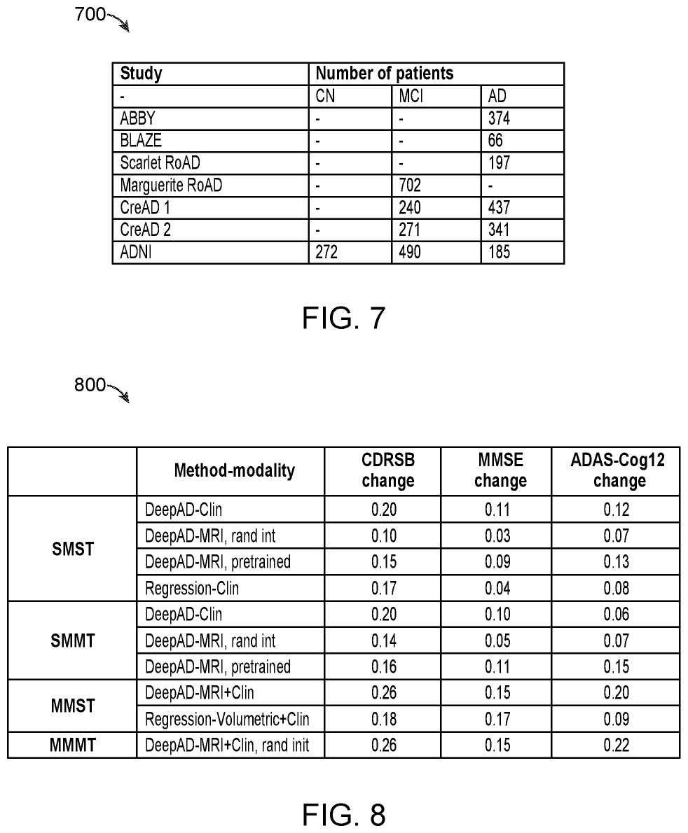

The performance of the machine learning model 120, consistent with implementations of the current subject matter, was tested based on a plurality of modalities for a plurality of patients. FIG. 7 depicts a table 700 summarizing the patient composition for the testing of the machine learning model 120. In the table 700, CN refers to cognitively normal patients, MCI refers to patients with mild cognitive impairment, and AD refers to patients with Alzheimer's disease. As described herein, the input clinical data (e.g., the clinical data 106) included demographic information (e.g., an age, sex, diagnosis, education level, and body mass index) of the patient, genomic information, one or more cognitive scores, such as CDRSB, MMSE, ADAS-Cog12, and FAQ. The input image (e.g., the image 108) included a raw three-dimensional magnetic resonance image and volumetric magnetic resonance imaging features. The raw three-dimensional magnetic resonance images were standardized by inferring a brain mask. During training of the machine learning model 120, the three-dimensional magnetic resonance images and segmentations were resampled isotropically to 1 mm voxel size, standardized to canonical (RAS+) orientation, with the intensity rescaled to 0.1, and with the Z-score normalized using only voxels inside the brain mask. Finally, the magnetic resonance image volumes were cropped or padded.

A weighted R² was defined separately for each endpoint of the machine learning model 120 by the weighted average of the R² on each dataset, as shown in Equation 3 below:

$${R}_{weighted - avg}^{2} = \frac{{\sum }_{\text{?}} {R}_{d}^{2} \times \vert d \vert}{{\sum }_{\text{?}} \vert d \vert} , \qquad Equation 3$$

$${R}^{2} = 1 - \frac{{SS}_{res}}{{SS}_{tot}}$$

$$\text{?} \text{indicates text missing or illegible when filed}$$

where SSᵣₑₛ and SSₜₒₜ respectively indicate the sum of squares of residuals and total sum of squares. This weighting guarantees that R² contributes to the weighted average R² in proportion to the dataset size.

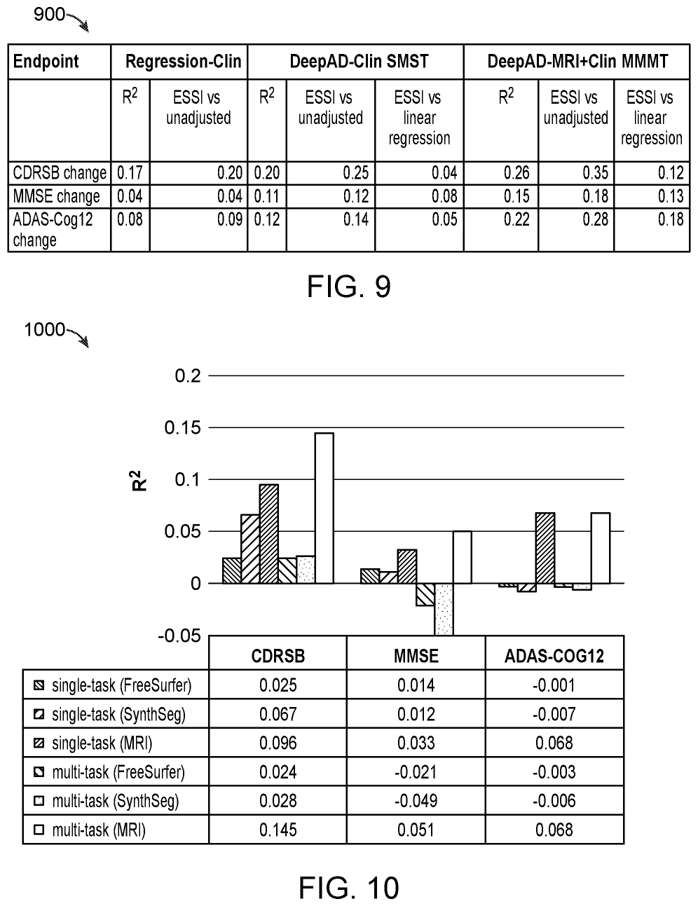

Effective Sample Size Increase (ESSI) was calculated for two setups: (1) comparing ESSI in a MMMT (multi-modality multi task modeling) adjusted analysis with respect to an unadjusted analysis (2) comparing ESSI in a MMMT adjusted analysis against a baseline linear regression model. FIG. 8 depicts a table 800 summarizing a performance comparison of machine learning models and approaches, based on a validation set, consistent with implementations of the current subject matter. For example, as shown in the table 800, the MMMT (e.g., the machine learning model 120) outperformed other models, such as single modality single task (SMST) models, single modality multi task (SMMT) models, and multi-modality single task (MMST) models). In the table 800, Clin refers to the clinical information modality (e.g., the clinical data 106) and MRI refers to the imaging modality (e.g., the image 108). FIG. 9 depicts a table 900 summarizing another performance comparison of the machine learning models and approaches, consistent with implementations of the current subject matter. In particular, the table 900 shows an ESSI comparison for the machine learning models shown in the table 800. As shown in the table 800 and the table 900, the machine learning model 120 exhibits the best performance when using all modalities to predict all three endpoints (e.g., the three cognitive scores). If used for covariate adjustment in a clinical trial, this shows that the machine learning model 120 may achieve up to a 35% increase in sample size compared to an unadjusted trail, or a 12% increase in sample size compared to a trial adjusted with the Regression Clin model shown in the table 900.

FIG. 10 depicts a graph 1000 illustrating a performance of a disease progression prediction system, consistent with implementations of the current subject matter. The graph 1000 shows the improved performance of the machine learning model 120 in predicting the progression of disease based on raw image inputs compared to extracted volumetric features, due at least in part to the encoded structure features included in the raw image inputs.

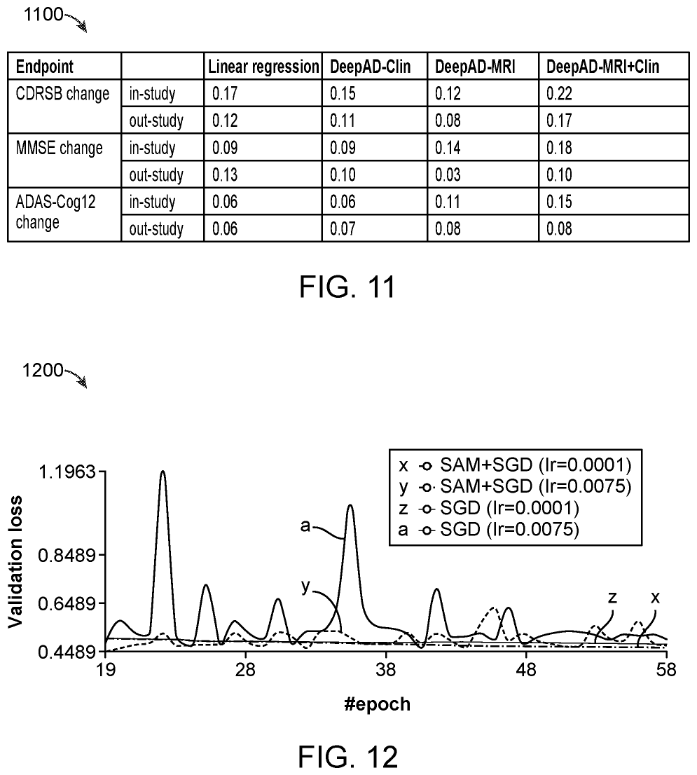

FIG. 11 depicts a table 1100 summarizing a performance comparison of machine learning models and approaches, consistent with implementations of the current subject matter. As shown in the table 1100, the robustness of the machine learning model 120 is evaluated by using two different external test sets including an in-study test set and an out-study test set. The trained multi-task machine learning model 120 was tested by using either clinical information or magnetic resonance images or both. As shown in the table 1100, the machine learning model 120 applied on both input modalities outperformed the other models significantly on in-study test sets. On the out-study test set, the machine learning model 120 applied on both modalities significantly outperformed the other models for predicting CDRSB, while reaching a comparable performance for MMSE and ADAS-COG12 prediction when applied only on clinical information. Moreover, stratifying patient cognitive trajectories by their predicted CDSB change reveals marked differences between groups, as the machine learning model 120 could separate stable from declining patients.

FIG. 12 depicts a graph 1200 illustrating a performance comparison of optimization approaches, consistent with implementations of the current subject matter. As shown in the graph 1200, application of the SAM optimization method leads to a more stable convergence and better minima compared to other optimization methods.

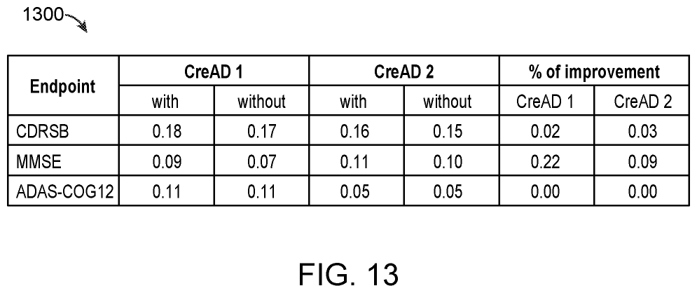

FIG. 13 depicts a table 1300 summarizing a performance comparison of machine learning models and approaches, consistent with implementations of the current subject matter. The table 1300 shows the effectiveness of the adversarial training of the domain detector 480 of the machine learning model 120. For example, as shown in the table 1300, incorporation of the adversarial loss in training the machine learning model 120 results up to 0.22% and 0.09% improvement in terms of R² for the MMSE cognitive score.

### Computing System

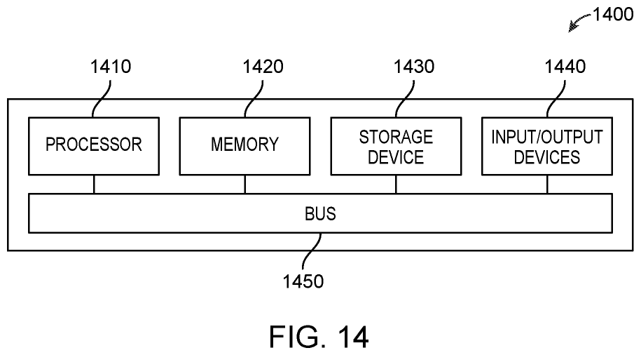

FIG. 14 depicts a block diagram illustrating a computing system 1400 consistent with implementations of the current subject matter. Referring to FIGS. 1-13, the computing system 1400 can be used to implement the machine learning controller 110, the machine learning model 120, the disease progression prediction system 100, and/or any components therein.

As shown in FIG. 14, the computing system 1400 can include a processor 1410, a memory 1420, a storage device 1430, and input/output devices 1440. The processor 1410, the memory 1420, the storage device 1430, and the input/output devices 1440 can be interconnected via a system bus 1450. The computing system 1400 may additionally or alternatively include a graphic processing unit (GPU), such as for image processing, and/or an associated memory for the GPU. The GPU and/or the associated memory for the GPU may be interconnected via the system bus 1450 with the processor 1410, the memory 1420, the storage device 1430, and the input/output devices 1440. The memory associated with the GPU may store one or more images described herein, and the GPU may process one or more of the images described herein. The GPU may be coupled to and/or form a part of the processor 1410. The processor 1410 is capable of processing instructions for execution within the computing system 1400. Such executed instructions can implement one or more components of, for example, the machine learning controller 110, the machine learning model 120, the disease progression prediction system 100, and/or the like. In some implementations of the current subject matter, the processor 1410 can be a single-threaded processor. Alternately, the processor 1410 can be a multi-threaded processor. The processor 1410 is capable of processing instructions stored in the memory 1420 and/or on the storage device 1430 to display graphical information for a user interface provided via the input/output device 1440.

The memory 1420 is a computer readable medium such as volatile or non-volatile that stores information within the computing system 1400. The memory 1420 can store data structures representing configuration object databases, for example. The storage device 1430 is capable of providing persistent storage for the computing system 1400. The storage device 1430 can be a floppy disk device, a hard disk device, an optical disk device, or a tape device, or other suitable persistent storage means. The input/output device 1440 provides input/output operations for the computing system 1400. In some implementations of the current subject matter, the input/output device 1440 includes a keyboard and/or pointing device. In various implementations, the input/output device 1440 includes a display unit for displaying graphical user interfaces.

According to some implementations of the current subject matter, the input/output device 1440 can provide input/output operations for a network device. For example, the input/output device 1440 can include Ethernet ports or other networking ports to communicate with one or more wired and/or wireless networks (e.g., a local area network (LAN), a wide area network (WAN), the Internet).

In some implementations of the current subject matter, the computing system 1600 can be used to execute various interactive computer software applications that can be used for organization, analysis and/or storage of data in various (e.g., tabular) format (e.g., Microsoft Excel®, and/or any other type of software). Alternatively, the computing system 1600 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1640. The user interface can be generated and presented to a user by the computing system 1600 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

1. A system, comprising:
a processor; and
a memory storing instructions which, when executed by the processor, result in operations comprising:
generating, by a machine learning model, a first feature representation based on clinical data associated with a baseline cognitive state of a patient;
generating, by the machine learning model, a second feature representation based on an image of a brain of the patient;
generating, by the machine learning model, a set representation by at least fusing the first feature representation and the second feature representation; and
predicting, by the machine learning model, a change in the baseline cognitive state over a time period based at least on the set representation.

2. The system of claim 1, wherein the fusing is performed using one or more fusion techniques including at least one of: concatenation, summation, simple attention, scaled dot product attention, applying a tensor fusion network, low rank fusion, and unidirectional contextual attention.

3. The system of claim 1, wherein the first feature representation is an encoded vector including a concatenation of at least one of a current cognitive score representing the baseline cognitive state of the patient, demographic information associated with the patient, and genomic information associated with the patient.

4. The system of claim 1, wherein the second feature representation includes at least one domain invariant embedded feature.

5. The system of claim 1, wherein the machine learning model includes a first machine learning model trained to generate the first feature representation; a second machine learning model trained to generate the second feature representation; a third machine learning model trained to generate the set representation; and a fourth machine learning model trained to predict the change in the baseline cognitive state over the time period.

6. The system of claim 1, wherein the machine learning model is trained, based at least on a plurality of modalities including the clinical data associated with the baseline cognitive state of the patient and the image of the brain of the patient.

7. The system of claim 1, wherein the machine learning model is pre-trained to predict the baseline cognitive state of the patient based at least on a plurality of brain images acquired at a plurality of time points and across a plurality of domains.

8. The system of claim 1, wherein the machine learning model is trained by at least adversarially training a domain detector of the machine learning model to reduce an inter-study domain shift associated with the image of the brain of the patient.

9. The system of claim 8, wherein the adversarially training includes adversarially training a feature extraction network of the machine learning model to learn domain invariant features for generating the second feature representation based at least on the image of the brain of the patient.

10. The system of claim 8, wherein the adversarially training includes applying a reverse gradient to the second feature representation to generate a domain detector input; and the adversarially training the domain detector is based at least on the domain detector input.

11. The system of claim 8, wherein the domain detector indicates a drift in the inter-study domain shift at inference.

12. The system of claim 1, wherein the change in the baseline cognitive state over time indicates a progression of Alzheimer's disease in the patient.

13. The system of claim 1, wherein the time period is 12 months.

14. The system of claim 1, wherein the image is a three-dimensional magnetic resonance imaging image including an inferred mask.

15. The system of claim 1, wherein the baseline cognitive state is represented by at least one cognitive score including at least one of a Clinical Dementia Rating Scale Sum of Boxes (CDRSB) score, an Alzheimer's disease Assessment Scale-Cognitive Subscale (ADAS-COG12) score, and a Mini-Mental State Examination (MMSE) score.

16. A computer-implemented method comprising:
generating, by a machine learning model, a first feature representation based on clinical data associated with a baseline cognitive state of a patient;
generating, by the machine learning model, a second feature representation based on an image of a brain of the patient;
generating, by the machine learning model, a set representation by at least fusing the first feature representation and the second feature representation; and
predicting, by the machine learning model, a change in the baseline cognitive state over a time period based at least on the set representation.

17. (canceled)

18. The method of claim 16, wherein the first feature representation is an encoded vector including a concatenation of at least one of a current cognitive score representing the baseline cognitive state of the patient, demographic information associated with the patient, and genomic information associated with the patient, and wherein the second feature representation includes at least one domain invariant embedded feature.

19. (canceled)

20. (canceled)

21. (canceled)

22. (canceled)

23. The method of claim 16, wherein the machine learning model is trained by at least adversarially training a domain detector of the machine learning model to reduce an inter-study domain shift associated with the image of the brain of the patient, the adversarial training includes
adversarially training a feature extraction network of the machine learning model to learn domain invariant features for generating the second feature representation based at least on the image of the brain of the patient,
applying a reverse gradient to the second feature representation to generate a domain detector input, and
adversarially training, based at least on the domain detector input, a domain detector to indicate a drift in the inter-study domain shift at inference.

24. (canceled)

25. (canceled)

26. (canceled)

27. (canceled)

28. (canceled)

29. The method of claim 16, wherein the image is a three-dimensional magnetic resonance imaging image including an inferred mask.

30. (canceled)

31. A non-transitory computer readable medium storing instructions,
which when executed by at least one data processor, result in operations comprising:
generating, by a machine learning model, a first feature representation based on clinical data associated with a baseline cognitive state of a patient;
generating, by the machine learning model, a second feature representation based on an image of a brain of the patient;
generating, by the machine learning model, a set representation by at least fusing the first feature representation and the second feature representation; and
predicting, by the machine learning model, a change in the baseline cognitive state over a time period based at least on the set representation.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)
